(19) United States
(12) Patent Application Publication
(10) Pub. No.: US 2015/0150545 A1
(43) Pub. Date: Jun. 4, 2015

(54) TONGUE DEPRESSOR WITH DETACHABLE SAMPLE COLLECTION DEVICE

(71) Applicant: Sakina O'Uhuru, Teaneck, NJ (US)

(72) Inventor: Sakina O'Uhuru, Teaneck, NJ (US)

(21) Appl. No.: 14/551,237

(22) Filed: Nov. 24, 2014

Related U.S. Application Data
(60) Provisional application No. 61/907,839, filed on Nov. 22, 2013.

(51) Int. Cl. A61B 13/00; A61F 13/38; A61B 10/02

(52) U.S. Cl. CPC A61B 13/00; A61B 10/02; A61F 13/38

(57) ABSTRACT

A tongue depressor has a first end and a second end and an intermediate portion. A sample collection device having a rod with a fibrous tip is mounted on the tongue depressor thereby facilitating collection of a sample from a patient's mouth using just one hand. The fibrous tip extends beyond the first end of the tongue depressor thereby facilitating collection of the sample. The rod can be easily removed from the tongue depressor for storage or analysis of the sample.

### CLAIM OF PRIORITY

This application claims the benefit of and priority to U.S. Provisional Patent Application No. 61/907,839, filed Nov. 22, 2014 and titled “TONGUE DEPRESSOR WITH DETACHABLE SAMPLE COLLECTION DEVICE” which application is incorporated herein by reference in its entirety.

### FIELD OF THE INVENTION

The present invention relates generally to the field of sample collection devices for diagnostic use and more particularly to a tongue depressor with a detachable sample collection device.

### BACKGROUND OF THE INVENTION

In the typical procedure for sample collection from the oral cavity the operator holds and manipulates a wooden tongue depressor to depress the patients tongue using one hand while holding and manipulating a sample collection device with the other hand. The sample collection device has a rod with a fibrous tip which is used to collect the sample.

During this procedure a nurse or a physician's assistant usually assists the operator by holding a flashlight to illuminate the oral cavity. After the desired sample is collected the sample collection device is inserted in a test tube containing a growth medium for storage and analysis. While this typical procedure is in widespread use it is both awkward and inconvenient for patients and especially for patients who are young children. Typical tongue depressors are generally rectangular with rounded ends and are traditionally made of wood. This results in a degree of unpleasantness for the patient due to the surface finish, taste and inadvertent unwanted contact between the edges of the tongue depressor and the patient's lips.

The general awkwardness of the typical sample collection procedure results in a degree of discomfort for the patent especially when the patient is a young child. There is therefore a need for improved equipment for sample collection from the oral cavity.

### OBJECTS AND SUMMARY OF THE INVENTION

It is an object of the present invention to provide a tongue depressor with a detachable sample collection device which can be operated with one hand to collect samples from the oral cavity of a patient.

Another object of the present invention is to provide a tongue depressor with a detachable sample collection device which can be used with conventional in vitro sample storage and transportation devices.

Another object of the present invention is to provide a tongue depressor with a detachable sample collection device in which the sample collection device can be easily detached from the tongue depressor.

Another object of the present invention is to provide a tongue depressor with a detachable sample collection device in which the tongue depressor has a relatively narrow intermediate portion to limit inadvertent contact with the patient's lips.

Yet another object of the present invention is to provide a tongue depressor with a detachable sample collection device which can be easily manufactured in volume resulting in a relatively low unit cost.

In accordance with the present invention there is provided a tongue depressor with a detachable sample collection device which, in a first embodiment, includes a sample collection device which is mounted on the tongue depressor via a dip formed on the tongue depressor. The sample collection device includes an elongated rod having a fibrous tip on a first end and a cap on the second end. The tip is proportioned to fit a test tube device for in vitro transportation of the sample after collection.

The tongue depressor has a first end which is rounded and a second end which includes a step portion and a dip which removably attaches the cap of the collection device to the tongue depressor. The step portion sets the longitudinal position of the collection device relative to the tongue depressor and also allows the rod to lie close to the top surface of the tongue depressor. The central portion of the tongue depressor is relatively narrow to limit contact with the patient's lips. The tongue depressor is proportioned to allow the fibrous tip to project beyond the first end to allow contact with cavity and facilitate collection of the sample.

In a second embodiment of the invention, the sample collection device includes an elongated rod having a first end and a second end. The first end has a fibrous tip. The second end is connected to the top surface of a tongue depressor via an adhesive layer or a welded area. The tongue depressor includes a first end a second end and an intermediate portion. The intermediate portion is relatively narrower than the first and second end portions. The fibrous end of the collection device projects beyond the first end of the tongue depressor.

An intermediate portion of the rod includes a notched portion which enables the collection device to be broken away from the tongue depressor for diagnostic study after the sample has been collected.

In a third embodiment of the invention, the sample collection device includes an elongated rod having a first end and a second end. The tongue depressor has a first end, a second end and an upper surface. The upper surface includes a longitudinal groove which is defined by a pair of wall portions. The elongated rod snaps into the groove potion and is removably retained by the wall portions. The fibrous end of the collection device extends beyond the first end of the tongue depressor.

In a fourth embodiment of the invention, the elongated rod of the collection device is retained by a pair of spaced apart walls which are formed on the surface of the tongue depressor.

In a fifth alternate embodiment of e invention, the rod and the tongue depressor are formed as a single unitary molded member. The rod has a first end portion and a second end portion. The second end portion is formed as a projection located on the top portion of the tongue depressor. There is a small gap between the intermediate portion of the rod and the top surface of the tongue depressor. An intermediate portion of the rod includes a notched portion thus enabling a user to easily separate the collection device from the tongue depressor.

The various advantages of the tongue depressor assemblies described herein include the ability to the collection device, once the non-swab end is placed in regular or angled apertures, slits or slots located on either end of the tongue depressor body, to pivot or move the collection device and swab tip in all directions (up/down, side to side, etc.) while the tongue depressor body remains in place on the patient's tongue or lower jaw. This capability allows the user to swab or take bodily fluid samples from a patient's mouth (or other bodily orifice) without having to move the depressor body. In most applications, such capability also allows for the user to use one hand to operate the tongue depressor assembly and use the other hand to shine a light source onto the patient's mouth when conducting the swabbing. In some other applications, the swab tip of the tongue depressor assembly may include medication (such as an antibiotic, a pain reliever or a pain numbing medication) for placement anywhere within the patient's mouth and the flexibility of movement of the collection device facilitates that task.

In another related advantage, the cost of manufacturing the tongue depressor assembly is lowered when the collection device is pre-mounted to the tongue depressor body via a perforated tab as compared to the sale of loose tongue depressors and pre-packaged swab sticks.

### BRIEF DESCRIPTION OF THE DRAWINGS

Other important objects and advantages of the present invention will be apparent from the following detailed description of the invention taken in connection with the accompanying drawings in which;

FIG. 1 is a side elevation view of a tongue depressor with a detachable sample collection device made in accordance with the present invention;

FIG. 2 is a top plan view of the tongue depressor of FIG. 1;

FIG. 3 is a cross-sectional view taken along the line 3-3 of FIG. 2;

FIG. 4 is a plan view of the sample collection device with the sample collection device shown removed from the tongue depressor of FIG. 1 and showing a test tube;

FIG. 5 is a side elevation view of a second embodiment of the tongue depressor of FIG. 1;

FIG. 6 is a top plan view of a second embodiment of the tongue depressor of FIG. 1;

FIG. 7 is a cross-sectional view taken along the line 7-7 of FIG. 6 showing an adhesive layer;

FIG. 8 is a cross-sectional view taken along the line 7-7 of FIG. 6 showing an alternative construction;

FIG. 9 is a top plan view of a third embodiment of the tongue depressor of FIG. 1;

FIG. 10 is a side elevation view of the third embodiment of the tongue depressor of FIG. 9;

FIG. 11 is a cross-sectional view taken along the line 11-11 of FIG. 9;

FIG. 12 is a cross-sectional view taken along the line 11-11 of FIG. 9 showing an alternative construction;

FIG. 13 is a top plan view of a fourth embodiment of the tongue depressor of FIG. 1;

FIG. 14 is a side elevation view of the fourth embodiment of the tongue depressor of FIG. 13;

FIG. 15 is a cross-sectional view taken along the line 15-15 of FIG. 13;

FIG. 16 is a cross-sectional view taken along the line 15-15 of FIG. 13 showing an alternative construction;

FIG. 17 is a top plan view of a fifth embodiment of the tongue depressor of FIG. 1;

FIG. 18 is a side elevation view of the embodiment of FIG. 17;

FIG. 19 is a cross-sectional view taken along the line 19-19 of FIG. 17;

FIGS. 20A-20D illustrate various views of a tongue depressor assembly with a detachable sample collection device made in accordance with the present invention; and

FIGS. 21A-21D illustrate various views of another tongue depressor assembly made in accordance with the present invention.

### DETAILED DESCRIPTION OF THE INVENTION

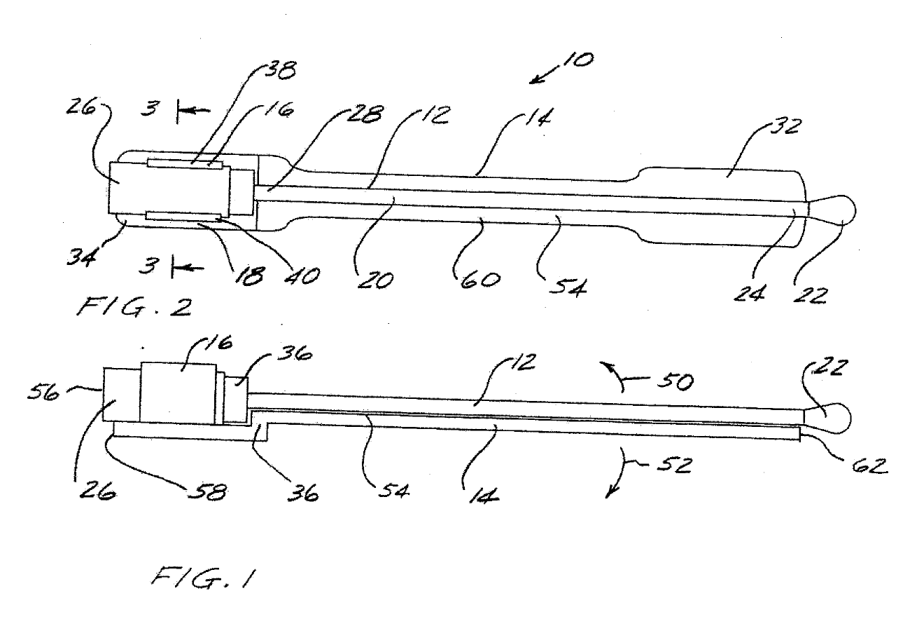

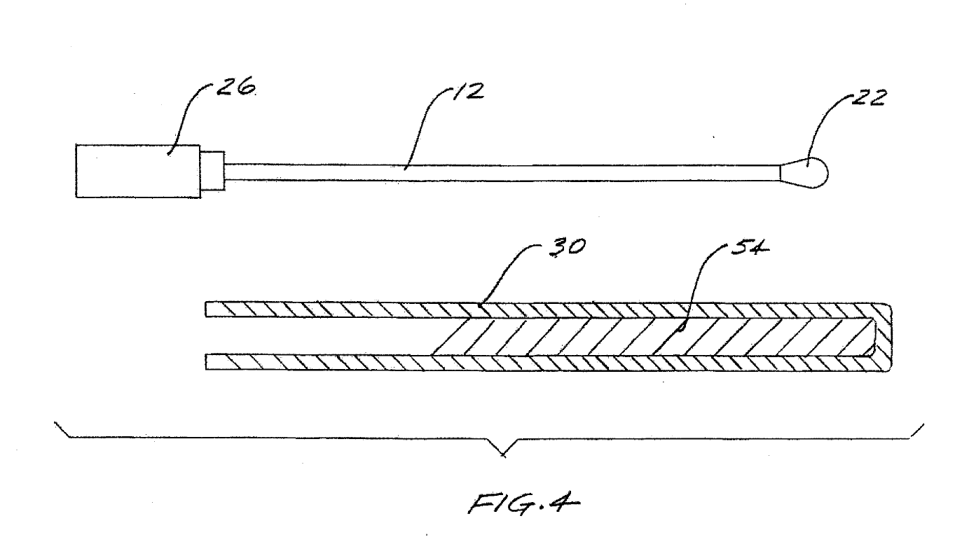

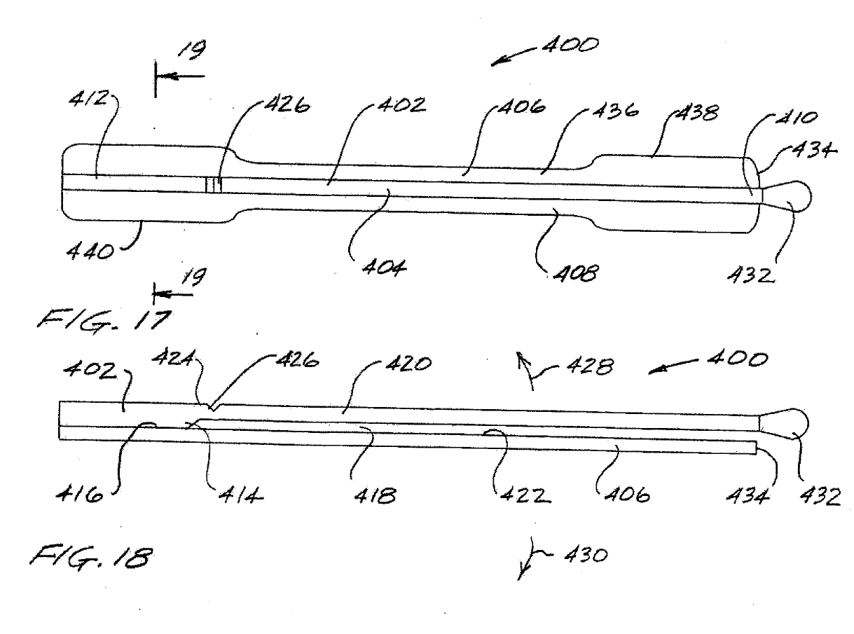

With reference to the drawings in which FIGS. 1-19 show a tongue depressor assembly 10 with a detachable sample collection device 12, made according to the present invention which, in a first embodiment, includes a sample collection device 12 which is removably mounted on a tongue depressor 14 via a clip 18 which is formed on the upper surface 18 of the tongue depressor 14. The sample collection device 12 includes an elongated rod 20 having a fibrous tip 22 on a first end 24 and a cap 28 on the second end 28. The cap 26 is generally cylindrical and is proportioned to a test tube device 30 for in vitro transportation of the sample after collection.

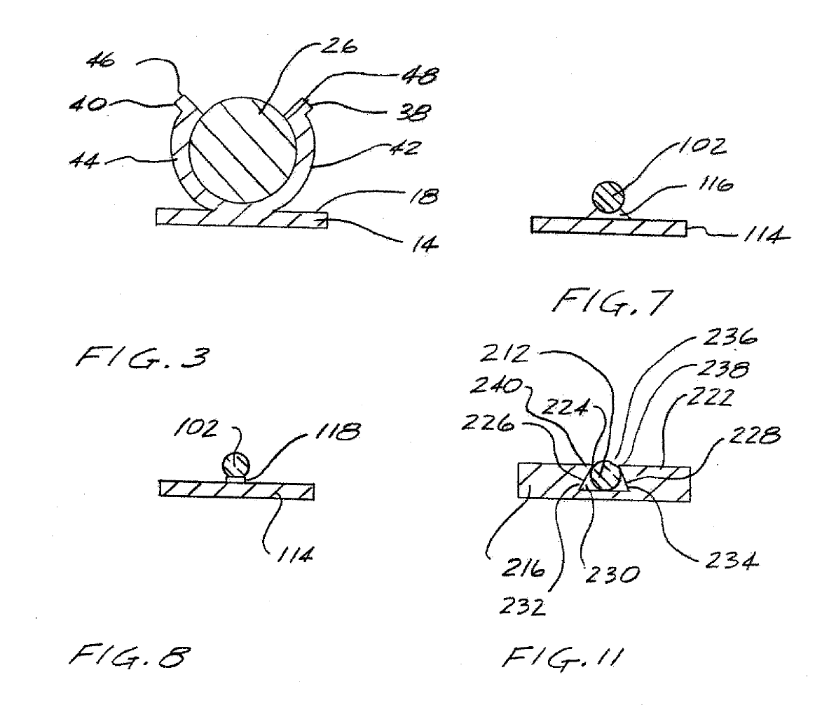

The tongue depressor 14 has a first end portion 32 which is rounded and a second end portion 34 which includes a step portion 36 and the clip 16 which removably attaches the cap 26 of the sample collection device 12 to the tongue depressor 14. The clip 16 includes a pair of arm portions 38, 40 which project from the upper surface 18 of the tongue depressor 14. The arm portions 38, 40 each have a semi-cylindrical portion 42, 44 and an outwardly directed end portion, as is shown in FIG. 3. The arms 38, 40 flex outwardly to accept the cylindrical portion 60 of the cap 26 which is disposed on the end 28 of the rod 20. After the sample has been collected on the fibrous tip 22 the cap 26 can be easily removed from the tongue depressor 14 by applying manual pressure in the directions shown by the arrows 50, 52 in FIG. 1. The specimen collection device 12 can be inserted in the test tube device 30 or in a vial which may have a growth medium 54 for storage, transportation and analysis.

The step portion 36 sets the longitudinal position of the collection device 12 relative to the tongue depressor 14 and also allows the rod 20 to lie close to the upper surface 54 of the tongue depressor 14. The end 56 of the cap 26 may protrude slightly beyond the end 58 of the tongue depressor 14 to aid in grasping the cap 26 during removal of the cap 26 from the clip 16. Alternatively, the end 54 of the cap 26 may be flush with the end 56 of the tongue depressor 14.

The central portion 60 of the tongue depressor 14 is narrower than the end portions 32, 34 to limit contact with the patient's lips during use. The tongue depressor 14 is proportioned to allow the fibrous tip 22 to project beyond the end 62 to allow contact with the oral cavity and efficient collection of the sample. The relatively narrow central portion 80 of the tongue depressor 14 allows the operator to conveniently reach and collect samples from various areas of the oral cavity which otherwise could be reached only with difficulty.

In a second embodiment of the Invention 100, shown in FIGS. 5-8, the sample collection device 102 includes an elongated rod 104 having a first end 106 and a second end 108 with the first end 106 having a fibrous tip 110. The second end 108 is connected to the top surface 112 of a tongue depressor 114 via an adhesive layer 116 as: shown in FIG. 7 or, alternatively, an ultrasonically welded area 118 as shown in FIG. 8. The tongue depressor 114 includes a first end portion 120 a second end portion 122 and an intermediate portion 124. The intermediate portion 124 is relatively narrower than the first and second end portions 120, 124. The fibrous tip 110 of the collection device 102 projects beyond the first end 126 of the tongue depressor 114, as previously described.

An intermediate portion 128 of the rod 104 includes a notched portion 130 which enables the collection device 102 to be easily broken away from the tongue depressor 114 for diagnostic use after the sample has been collected. Pulling the rod 104 and the tongue depressor 114 apart in the directions shown by the arrows 132, 134 in FIG. 5 easily separates the collection device 102 from the tongue depressor 114.

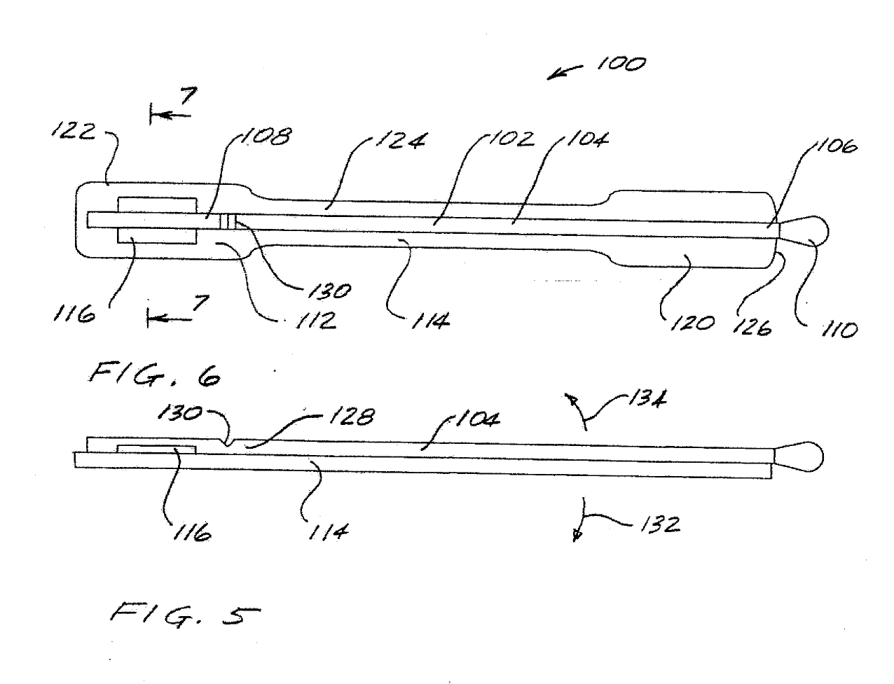

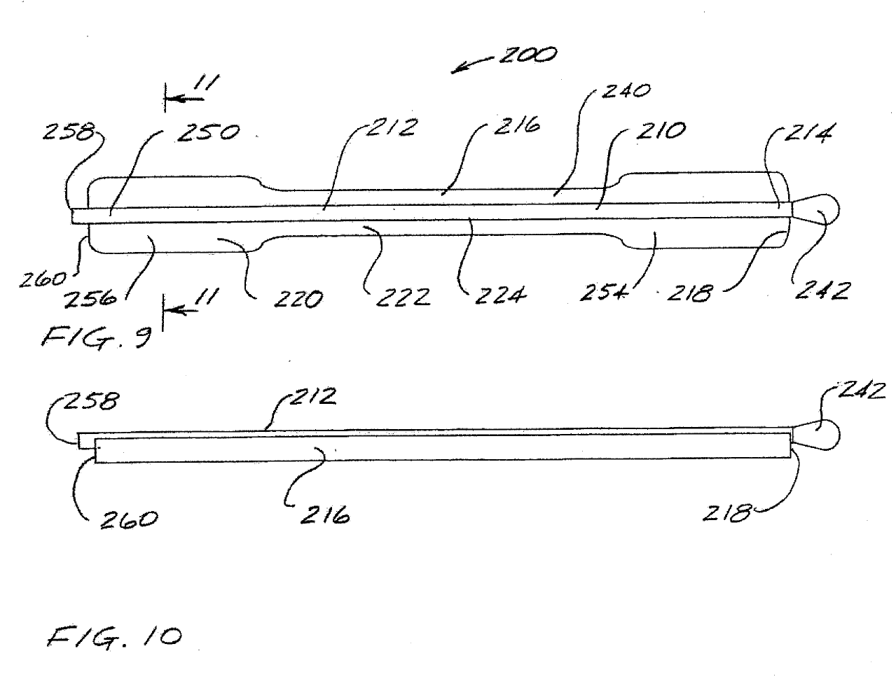

In a third embodiment of the invention 200 shown in FIGS. 9-12 the sample collection device 210 includes an elongated rod 212 having an end 250 and a second end 214. As previously described, the tongue depressor 216 has a first end 218, a second end 220 and an upper surface 222, The upper surface 222 includes a longitudinal groove 224 which is defined by a pair of spaced apart wall portions 226, 228. As Is best shown In FIG. 11, the space 230 between the lower portions 232, 234 of the wall portions 226, 228 is greater than the space 236 between the upper portions 238, 240 of the wall portions 226, 228 thereby enabling the rod 212 to snap fit into the groove 224. The elongated rod 212 snaps into the groove 224 and is removably retained by the wall portions 226, 228, A fibrous tip 242 on the end 214 of the rod 212 extends beyond the first end 218 of the tongue depressor 216.

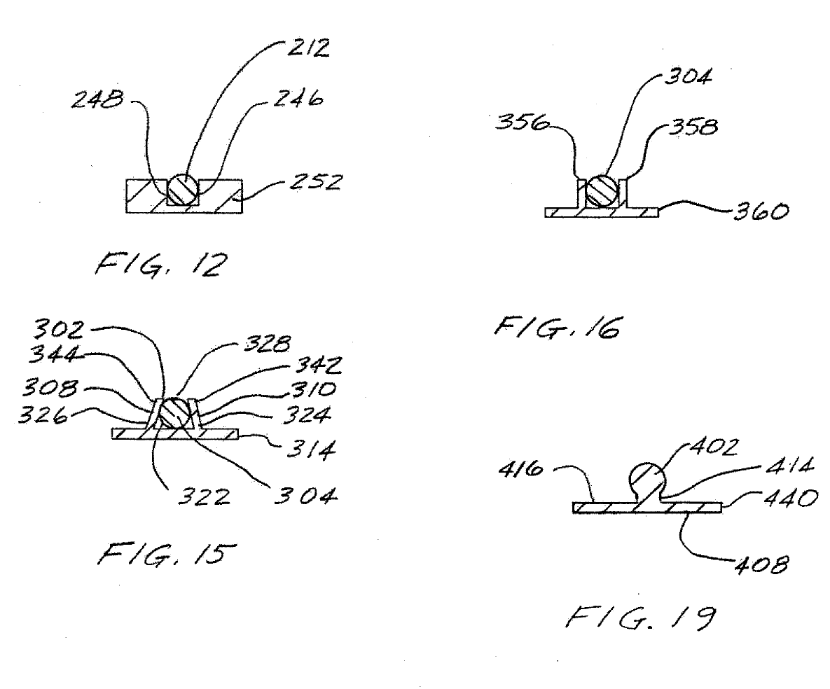

Alternatively, the spaced apart wall portions 246, 248 of the tongue depressor 252 may be parallel and also generally perpendicular to the surface 254 of the tongue depressor 252. The wall portions 246, 248 closely fit the elongated rod 212 and retain the elongated rod 212 as is shown in FIG. 12. The elongated rod 212 and the wall portions 246, 248 are proportioned to allow the elongated rod 212 to snap fit between the wall portions 246, 248.

An intermediate portion 240 of the tongue depressor 218 is relatively narrower than the end portions 254, 258 to reduce contact between the tongue depressor 216 and the patient's lips during use.

The end 258 of the rod 212 extends beyond the end 260 of the tongue depressor 216 to facilitate grasping the rod 212 and separating the rod 212 from the tongue depressor 216.

A fourth embodiment of the invention 300 is shown in FIGS. 13-16 and is similar to the third embodiment of the invention 200 with the exception that the groove 302 which retains the rod 304 which is part of the collection device 306 is defined by a pair of wall portions 308, 310 which project from the upper surface 312 of the tongue depressor 314. The rod 304 has a first end 316 and a second end 318 with a fibrous tip 320 disposed on the first end 316. As is shown in FIG. 15, the space 322 between the lower portions 324, 326 of the wall portions 308, 310 is greater than the space 328 between the upper portions 342, 344 of the wall portions 308, 310 to enable the rod 304 to snap fit into the groove 302.

The wall portions 308, 310 significantly increase the resistance to bending of the tongue depressor 314 in the directions shown by the arrows 346, 348 in FIG. 14 enabling the intermediate portion 350 of the tongue depressor 314 to be significantly narrower than the end portions 352, 354 and yet retain sufficient resistance to bending. This narrow intermediate portion 350 results in a reduced likelihood of contact with the patient's lips during use and an economy in material used during manufacture.

Alternatively, the spaced apart wall portions 356, 358 of the tongue depressor 360 may be may be parallel and also generally perpendicular to the surface 364 of the tongue depressor 360. The wall portions 356, 358 closely fit the elongated rod 304 and retain the elongated rod 304 as is shown in FIG. 16. The elongated rod 304 and the wall portions 356, 358 are proportioned to allow the elongated rod 304 to snap fit between the wall portions 356, 358.

The end 366 of the rod 304 extends beyond the end 388 of the tongue depressor 360 to facilitate grasping the rod 304 and separating the rod 304 from the tongue depressor 360 as is shown in FIG. 14.

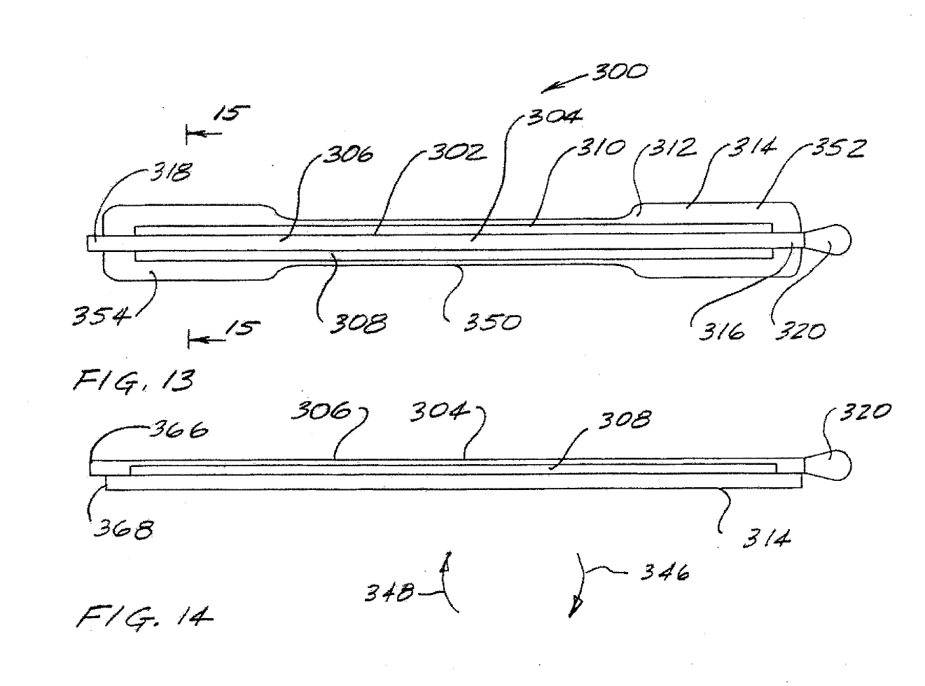

In a fifth alternate embodiment of the invention 400 shown in FIGS. 17-19 the rod 402 which is part of the collection device 404 and the tongue depressor 406 are formed as a single unitary molded member 408. The rod 402 has a first end portion 41.0 and a second end portion 412. The second end portion 412 has a relatively thin projecting portion 414 disposed between the rod 402 and the top portion 416 of the tongue depressor 406. The rod 402, the projection 414 and the tongue depressor 408 form the unitary member 408. There is a small gap 418 between the intermediate portion 420 of the rod 402 and the top surface 422 of the tongue depressor 408. An intermediate portion 424 of the rod 402 includes a notched portion 426 thus enabling a user to separate the collection device 404 from the tongue depressor 406 by pulling in the directions shown by the arrows 428, 430 in FIG. 18 after the sample has been collected. The end 410 of the rod 402 has a fibrous tip 432 which extends beyond the end 434 of the tongue depressor 406, the intermediate portion 436 of the tongue depressor 408 is relatively narrower than the end portions 438, 440 as previously described.

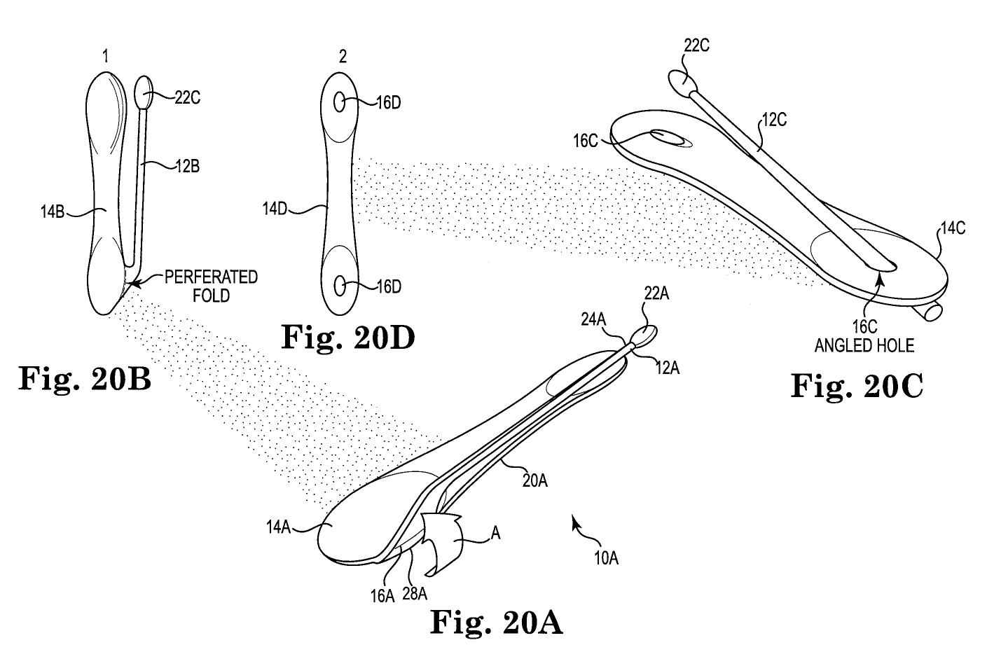

Referring now to FIGS. 20A-20D, there is illustrated various views of another example tongue depressor assembly 10A with a detachable sample collection device 12A made in accordance with the present invention. In this example embodiment, detachable sample collection device 12A is pre-mounted on a tongue depressor body 14A and removable via a tab or fold 16A that is perforated and turns towards direction of arrow to detach itself from depressor body 14A. The sample collection device 12A includes an elongated rod 20A having a fibrous tip 22A on a first end 24A and a second end 28A. Similar components and elements are illustrated in the related figures including regular or angled apertures 16C and 16D for insertion of one end of collection device 12A-12C to allow for pivoting of the collection device in all directions (up/down, side to side, etc.). This capability allows the user to swab or take bodily fluid samples from a patient's mouth (or other bodily orifice) without having to move depressor body 14A-14D. In one related embodiment, tab 16A serves as a stop for device 12A-12D to prevent it from slipping all the way through. An optional cap can be included as part of the assembly that is generally cylindrical and is proportioned to a test tube device for in vitro transportation of the sample after collection.

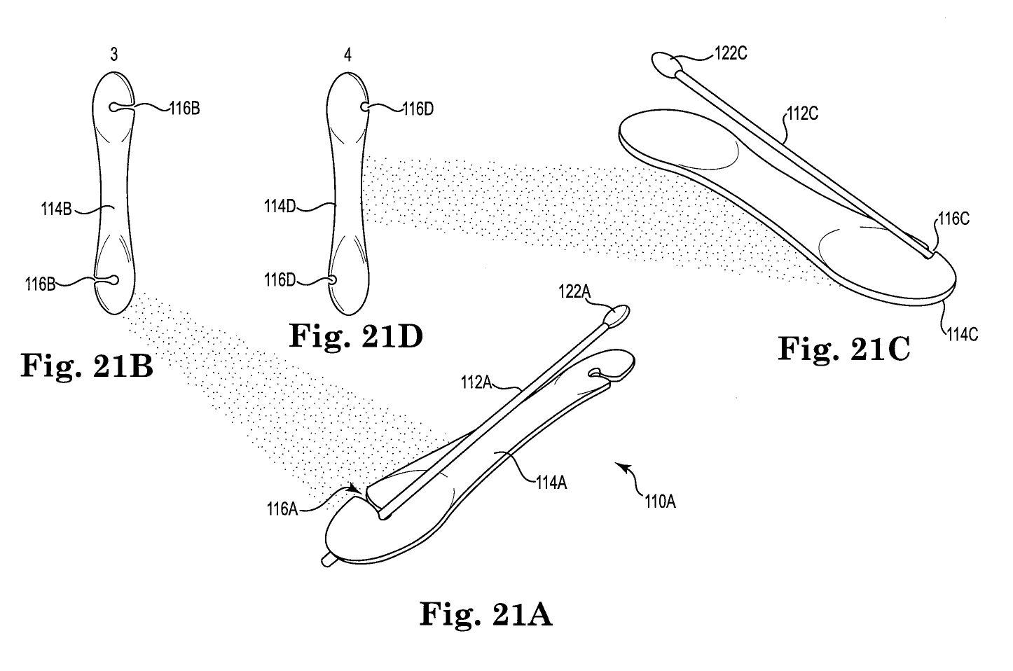

FIGS. 21A-21D illustrate various views of another tongue depressor assembly 110A made in accordance with the present invention. In this example embodiment, a sample collection device 112A is removable and temporarily mounted on a tongue depressor body 114A via a slot 116A and turns from side to side and up and down. The sample collection device 112A includes an elongated rod 120A having a fibrous tip 122A on a first end 124A and a second end 128A. Similar components and elements are illustrated in the related figures including regular or angled slits or slots 16C and 16D for insertion of one end of collection device 112A-112C to allow for pivoting of the collection device in all directions. This capability allows the user to swab or take bodily fluid samples from a patient's mouth (or other bodily orifice) without having to move depressor body 114A-114D. In one related embodiment, a tab is included at one end to serve as a stop for device 112A-112D to prevent it from slipping all the way through the slots. An optional cap can be included as part of the assembly that is generally cylindrical and is proportioned to a test tube device for in vitro transportation of the sample after collection.

The foregoing specific embodiments of the present invention as set forth in the specification herein are for illustrative purposes only. Various deviations and modifications may be made within the spirit and scope of the invention without departing from the main theme thereof.

1-16. (canceled)

17. A tongue depressor assembly comprising:
a sample collection device including a rod having extended length with a first end and a second end, the rod further including a fibrous tip mounted on the first end of the extended rod; and
a tongue depressor having a first end, a second end and an intermediate portion, the tongue depressor including at least one aperture that extends from a top surface to a bottom surface of the tongue depressor, the at least one aperture adapted to receive the second end of the rod.

18. The tongue depressor assembly of claim 17 wherein the tongue depressor assembly is configured to allow a user to move the fibrous tip above a surface of the tongue depressor while the tongue depressor is in contact with a patient's tongue.

19. The tongue depressor assembly of claim 18 wherein the aperture is configured to prevent the fibrous tip from passing through.

20. The tongue depressor assembly of claim 18 wherein the at least one aperture includes a slit disposed substantially in the intermediate portion of the tongue depressor.

21. The tongue depressor assembly of claim 18 wherein the at least one aperture includes a slit disposed in the first end or the second end of the tongue depressor.

22. The tongue depressor assembly of claim 18 wherein the at least one aperture includes at least one angled wall configured to be angled towards the first end of the tongue depressor.

23. The tongue depressor assembly of claim 22 wherein the at least one aperture includes at front angled wall and second angled wall, both walls configured to be angled towards the first end of the tongue depressor.

24. The tongue depressor assembly of claim 18 wherein the at least one aperture is disposed on a side wall of either the first end or the second end of the tongue depressor.

25. The tongue depressor assembly of claim 18 further comprising connection means for attaching the rod to the tongue depressor.

26. The tongue depressor assembly of claim 18 further comprising a removable cap disposed on the second end of the rod configured to cover the fibrous tip after use on a patient.

27. The tongue depressor assembly of claim 18 wherein the intermediate portion of the tongue depressor is relatively narrower than either of the first end and the second end portions.

28. A tongue depressor assembly comprising:
a sample collection device including a rod having extended length with a first end and a second end, the rod further including a fibrous tip mounted on the first end of the extended rod; and
a tongue depressor having a first end, a second end and an intermediate portion, the tongue depressor including at least one aperture that extends from a top surface to a bottom surface of the tongue depressor, the at least one aperture adapted to receive the second end of the rod, wherein the at least one aperture includes a slit disposed substantially in the intermediate portion of the tongue depressor and wherein the aperture is configured to prevent the fibrous tip from passing through.

29. The tongue depressor assembly of claim 28 wherein the tongue depressor assembly is configured to allow a user to move the fibrous tip above a surface of the tongue depressor while the tongue depressor is in contact with a patient's tongue.

30. The tongue depressor assembly of claim 29 wherein the slit includes at least one angled wall configured to be angled towards the first end of the tongue depressor.

31. A bodily tissue depressor assembly comprising:
a sample collection device including a rod having extended length with a first end and a second end, the rod further including a fibrous tip mounted on the first end of the extended rod; and
a tissue depressor having a first end, a second end and an intermediate portion, the tissue depressor including at least one aperture that extends from a top surface to a bottom surface of the tissue depressor, the at least one aperture adapted to receive the second end of the rod.

32. The bodily tissue depressor assembly of claim 31 wherein the tissue depressor assembly is configured to allow a user to move the fibrous tip about a bodily orifice while the depressor is in contact with a patient's bodily tissue adjacent the orifice.

33. The bodily tissue depressor assembly of claim 32 wherein the aperture is configured to prevent the fibrous tip from passing through.

34. The bodily tissue depressor assembly of claim 32 wherein the at least one aperture includes a slit disposed substantially in the intermediate portion of the tissue depressor.

35. The tongue depressor assembly of claim 32 wherein the at least one aperture includes a slit disposed in the first end or the second end of the tissue depressor.

36. The bodily tissue depressor assembly of claim 32 wherein the at least one aperture includes at least one angled wall configured to be angled towards the first end of the tissue depressor.
